(19) United States
(12) Patent Application Publication
(10) Pub. No.: US 2011/0092763 A1
(43) Pub. Date: Apr. 21, 2011

(54) Methods for Embryo Characterization and Comparison

(75) Inventors: Matthew Rabinowitz, Portola Valley, CA (US); David S. Johnson, San Francisco, CA (US); Nigam Shah, San Jose, CA (US); George Gemelos, San Jose, CA (US)

(73) Assignee: Gene Security Network, Inc.

(21) Appl. No.: 12/994,260

(22) PCT Filed: May 27, 2009
(86) PCT No.: PCT/US09/45335

§ 371 Date: Dec. 20, 2010

Related U.S. Application Data
(60) Provisional application No. 61/128,961, filed on May 27, 2008.
(60) Provisional application No. 61/188,343, filed on Aug. 8, 2008.

(51) Int. Cl. A61B 17/435; G06F 19/00

(52) U.S. Cl. 600/34; 702/19

(57) ABSTRACT

Disclosed herein are methods for determining which embryos from a group of embryos are most likely to implant and develop as desired. In an embodiment of the present disclosure, one or more cells are biopsied from each of the embryos, and the genetic condition of those cells are determined. Within a group of embryos that each test positive for aneuploidy, the likelihood that each embryo contains euploid cells may be determined from the type of aneuploidy observed in the biopsied cells. This knowledge may be used to make a decision as to which embryos to transfer to a uterus. In an embodiment of the present disclosure, these determinations are made for the purpose of embryo selection in the context of in vitro fertilization.

### FIELD

The present disclosure relates generally to the field of acquiring, manipulating high fidelity genetic data for medically predictive purposes.

### BACKGROUND

In 2006, across the globe, roughly 800,000 in vitro fertilization (IVF) cycles were run. Of the nearly 130,000 cycles run in the US, about 10,000 involved pre-implantation genetic diagnosis (PGD). Current PGD techniques are unregulated, expensive and can be unreliable: error rates for screening disease-linked loci or aneuploidy are on the order of 10%, each screening test costs roughly $5,000, and the likelihood of an IVF cycle resulting in a live birth of a healthy baby is typically lower than 50%, and can be much lower for women of advanced age, or with medical issues. There is a great need for an affordable technology that can better determine which embryos are more likely to implant, and result in a successful pregnancy.

The process of PGD during IVF currently involves biopsy of embryos generated using assisted conception techniques. There are two potential sources of embryonic genetic material for PGD aneuploidy screening: one (or sometimes two) blastomeres from cleavage stage embryos (typically day 3 post-fertilization) or several (typically 4-10) tropechtoderm cells from blastocyst stage embryos (typically day 5 post-fertilization). Using cleavage stage single cell biopsy is the most common approach to PGD. Isolation of single cells from human embryos, while highly technical, is now routine in IVF clinics. Polar bodies, blastomeres, and tropechtoderm cells have been isolated with success. However, there is only a limited amount of time available for preimplantation testing—most clinics aim to transfer the embryos to the mother within 32 hours of biopsy. Consequently, diagnostic methods must be rapid as well as accurate.

Normal humans have two sets of 23 chromosomes in every diploid cell, with one set originating from each parent. Aneuploidy, (i.e., the state of a cell with extra or missing chromosome(s), and uniparental disomy, the state of a cell with two of a given chromosome both of which originate from one parent), is believed to be responsible for a large percentage of failed implantations and miscarriages, and some genetic diseases. When only certain cells in an individual are aneuploid, the individual is said to exhibit mosaicism.

The most common reason that embryos fail to carry to term is that they are aneuploid or mosaic. This can result in the embryo failing to implant, or can result in a spontaneous abortion. Detection of chromosomal abnormalities can identify individuals or embryos with conditions such as Down syndrome, Klinefelter's syndrome, and Turner syndrome, among others, and potentially increase the chances of a successful pregnancy. Testing for chromosomal abnormalities is especially important as the age of a potential mother increases: between the ages of 35 and 40 it is estimated that between 40% and 70% of the embryos are abnormal, and above the age of 40, between 50% and 80% of the embryos are likely to be abnormal. In cases where, during an IVF cycle, all of the embryos test positive for aneuploidy, physicians may randomly choose a few embryos to implant, hoping that one or more of the embryos will implant and develop as desired. Typically, IVF practitioners try to avoid the negative potential of aneuploidy by only transferring embryos from which a biopsied cell has tested euploid at all tested chromosomes. There is a great need for a method that can determine which embryos, of a group of embryos that all test positive for aneuploidy, are more or less likely to implant and result in the birth of a healthy baby.

The traditional method for determining ploidy state is karyotyping, which involves the isolation of a single cell, the staining of the chromosomes in that cell, and the visualization and identification of the chromosomes. A major drawback to karyotyping is the high cost. Currently, the most common method for determining ploidy state of a blastomere is fluorescent in situ hybridization (FISH) which can determine large chromosomal aberrations and polymerase chain reaction (PCR)/electrophoresis, and which can determine the identity of a small number of SNPs or other alleles. FISH involves the chromosome-specific hybridization of fluorescently tagged probes to cellular DNA, and subsequent visualization and quantification of the amount of fluorescent probes present. The technique is complex and expensive enough that generally only a small selection of chromosomes are tested. This results in a significant risk of misdiagnosis as some embryos may be aneuploid for chromosomes that were not analyzed. In addition, FISH has a low level of specificity. Roughly seventy-five percent of PGD today measures high-level chromosomal abnormalities such as aneuploidy, using FISH, with error rates on the order of 10-15%.

While aneuploidy is a universally negative state, it is possible for mosaic embryos to self-correct, presumably through attrition of aneuploid cells and the concurrent development of euploid cells. The mechanism of mosaicism in human IVF embryos is currently not understood, nor is it understood how to use a model for mosaicism, together with determination of different kinds of aneuploidies in one or multiple blastomeres, to predict the state of unmeasured cells in an embryo. There is a great need for a method that can predict which embryos that test positive for aneuploidy may be more or less likely to contain euploid cells, and consequently may develop as desired. There are no methods described in the art that can statistically determine which embryos, from which at least one cell as tested positive for aneuploidy, are more or less likely to develop as desired. There is a great need for a method which could differentiate embryos that test positive for aneuploid cells into those which are more or less likely to be a mosaic, and thus possibly self-correcting, embryo, as opposed to an aneuploid embryo.

Most embryos affected by aneuploidy develop from gametes with meiosis I or meiosis II nondisjunction errors; these meiotic errors give rise to aneuploid embryos which are very unlikely to self-correct and lead to a healthy birth. Aneuploidy resulting from mitotic errors often results in mosaic embryos, which have a much higher likelihood of self-correction. Aneuploidy in born children is a common and universally unacceptable clinical outcome linked to meiotic errors; consequently, there is a great need for differentiating meiotic from mitotic errors.

### SUMMARY

Methods of embryo characterization and comparison are disclosed herein. According to aspects illustrated herein, there is provided a method for comparing embryos, the method including: obtaining one or more cells from each embryo in a set of embryos; determining one or more subcharacteristics of one or more characteristics of each obtained cell; and estimating a likelihood that each embryo will develop as desired, based on the one or more subcharacteristic of the one or more cells which were obtained from that embryo.

According to aspects illustrated herein, there is provided a method of characterizing an embryo for insertion into a uterus, the method including: selecting at least one characteristic; determining a first subcharacteristic of the at least one characteristic of at least one cell from an embryo; using the determined first subcharacteristic, predicting a probability of a second cell from the embryo having a second subcharacteristic; and characterizing the embryo based on the predicted probability.

In an embodiment of the present disclosure, the method is used to determine which embryos have the best chance of developing into healthy babies if those embryos are transferred to a receptive uterus. In an embodiment of the present disclosure, the method is used to increase implantation rates, and thus possibly decreasing the number of IVF cycles necessary to achieve a successful pregnancy. In an embodiment of the present disclosure, the method provides a means to group the embryos into groups, wherein each group is defined by at least one subcharacteristic, each group may contain zero, one or more embryos, and wherein the likelihood that each embryo in a particular group will develop as desired is estimated based on the at least one subcharacteristic. In an embodiment of the present disclosure, the method provides a means to relatively characterizing the embryos. In this embodiment, the relative characterization may include ranking the embryos based on the estimated likelihood of that embryo developing as desired. In this embodiment, once relative probabilities have been determined, embryos can be ranked, and a more informed choice can be made as to which embryos to transfer. In an embodiment, the relative characterization of embryos may include ranking the embryos based on the estimated likelihood of that embryo developing as desired. In an embodiment, the ranking may be performed to select at least one embryo to insert into a uterus. In an embodiment, the method further comprises inserting an embryo into a uterus.

In an embodiment, the present disclosure provides a method that may determine which embryos are more or less likely to result in the birth of a healthy baby, based on one or more characteristics of the embryo. This may be done by categorizing embryos into different groups, or ‘bins’, where those groups have statistically different chances of developing as desired and resulting in a successful pregnancy. The bins may then be ranked by probability, and by transferring the embryos calculated to be most likely to develop as desired, an IVF clinician can maximize the chance that an IVF patient will have a healthy baby as a result of a given IVF cycle. In an embodiment, some of the characteristics used for making decisions regarding transfer of embryos may include embryo morphology, the presence or absence of aneuploidy, and the presence or absence of one or more disease-linked genes. In an embodiment, the method may be employed to rank embryos by grouping different types of aneuploidy that correlate with higher and lower potential implantation rates. In an embodiment, the type of aneuploidy may be a characteristic used to group embryos.

There are three types of cell divisions where non-disjunction in progenitor cells could give rise to abnormal daughter cells: (i) meiosis I, (ii) meiosis II, and (iii) mitosis. Because gametes are the founder cells of the embryo, meiosis I/II errors usually result in uniformly aneuploid embryos, unless a correction event occurs during further development. The main cause of aneuploidy is nondisjunction during meiosis. Maternal nondisjunction constitutes 88% of all nondisjunction, of which 65% occurs in meiosis 1 and 23% in meiosis II. Common types of human aneuploidy include trisomy from meiosis I nondisjunction, monosomy, and uniparental disomy. In a particular type of trisomy that arises in meiosis II nondisjunction, or M2 trisomy, an extra chromosome is identical to one of the two normal chromosomes. M2 trisomy (also called mitotic trisomy) is particularly difficult to detect. Implantation of these embryos leads to universally undesired outcomes such as failed embryo implantation, miscarriage, or birth of a trisomic offspring.

Mitotic errors, on the other hand, usually lead to formation of mosaic embryos where an extra chromosome (trisomy) in one daughter cell is frequently associated with a lost chromosome (monosomy) in another cell. Assuming that a genetic recombination event occurs during meiosis, both types of meiotic errors (associated with true aneuploidy) can be distinguished from mitotic errors (associated with mosaicism) based on whether the chromosomes are ‘matched’ or ‘unmatched’. Specifically, meiotic disjunction errors will give rise to ‘unmatched’ chromosome copy errors whereas post-fertilization mitotic disjunction errors will give rise to ‘matched’ chromosome copy errors since crossovers do not occur during post-fertilization cell division.

Current PGD methods such as FISH cannot distinguish meiosis I/II errors from mitotic errors, and although embryo mosaicism can sometimes be distinguished from true aneuploidy when at least two blastomeres are analyzed, it is not guaranteed. Additionally, there is a potentially detrimental effect of a 2-cell biopsy on a 3-day embryo's development. In some embodiments of the disclosure, this effect may be avoided using the method which may infer the probable ploidy state of the embryo's other cells based on single cell measurements.

In an embodiment, the present disclosure may provide a method to distinguish meiosis I/II errors from mitotic errors, and to use this knowledge to rank the embryos by the likelihood that they will implant and carry to term.

The present disclosure may employ mathematical correlations between the likelihood of an embryo to implant and carry to term and aneuploidy characteristics identified in a specific embryo. Such aneuploidy characteristics may include the parental origin of a trisomy, the identity of the aneuploid chromosome, and/or the number of aneuploid chromosomes in a cell. An embodiment may use a wide range of additional correlations to differentiate and rank embryos based on their likelihood to implant and carry to term.

The systems, methods, and techniques of the present disclosure may be used in conjunction with embryo screening in the context of IVF, or prenatal testing procedures, in the context of non-invasive prenatal diagnosis. The systems, methods, and techniques of the present disclosure may lead to increasing the probability that the embryos generated by in vitro fertilization are successfully implanted. The embodiments of the present disclosure may also be used to increase the probability that an implanted embryo is carried through the full gestation period, and result in the birth of a healthy baby. In some embodiments, the systems, methods, and techniques of the present disclosure may be employed to decrease the probability that the embryos and fetuses obtained by in vitro fertilization and are implanted and gestated are at risk for a chromosomal, congenital or other genetic disorder.

Various embodiments provide certain advantages. Not all embodiments of the disclosure share the same advantages and those that do may not share them under all circumstances. Further features and advantages of the embodiments, as well as the structure of various embodiments are described in detail below with reference to the accompanying drawings.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 shows an embodiment of a statistical model for the creation of mosaicism;

FIG. 2 shows embodiments of meiosis I nondisjunction, Meoisis II nondisjunction and mitotic errors;

FIG. 3 shows embodiments of CDF plots for chromosomes under disomy and unmatched trisomy;

FIG. 4 shows embodiments of mean improvement in implantation rates using a model in accordance with the present disclosure;

FIG. 5 shows embodiments of a histogram of improvement in rates of normal embryo selection;

FIG. 6 shows embodiments of a mean improvement in implantation rates using internal data; and

FIG. 7 shows embodiments of a probability of a blastomere being diploid based on ploidy state of biopsied cell.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The embodiments of the present disclosure are not all limited in its application to the details of construction and the arrangement of components set forth in the following description or illustrated in the drawings. Embodiments of the present disclosure are capable of being arranged in other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the present disclosure are described below with reference to illustrative embodiments. It should be understood that reference to these illustrative embodiments is not made to limit aspects of the present disclosure in any way. Instead, illustrative embodiments are used to aid in the description and understanding of various aspects of the present disclosure. Therefore, the following description is intended to be illustrative, not limiting.

The embodiments of the present disclosure may include a method for comparing embryos including: obtaining one or more cells from each embryo in a set of embryos; determining one or more subcharacteristic of each obtained cell; and estimating the likelihood that each embryo will develop as desired, based on the one or more subcharacteristic of the one or more cells which were obtained from the embryo. The embodiments of the present disclosure may include a method of characterizing an embryo for insertion into a uterus including: selecting at least one characteristic; determining a first subcharacteristic of the at least one characteristic of at least one cell from an embryo; using the determined first subcharacteristic, predicting a probability of a second cell from the embryo having a second subcharacteristic; and characterizing the embryo based on the predicted probability.

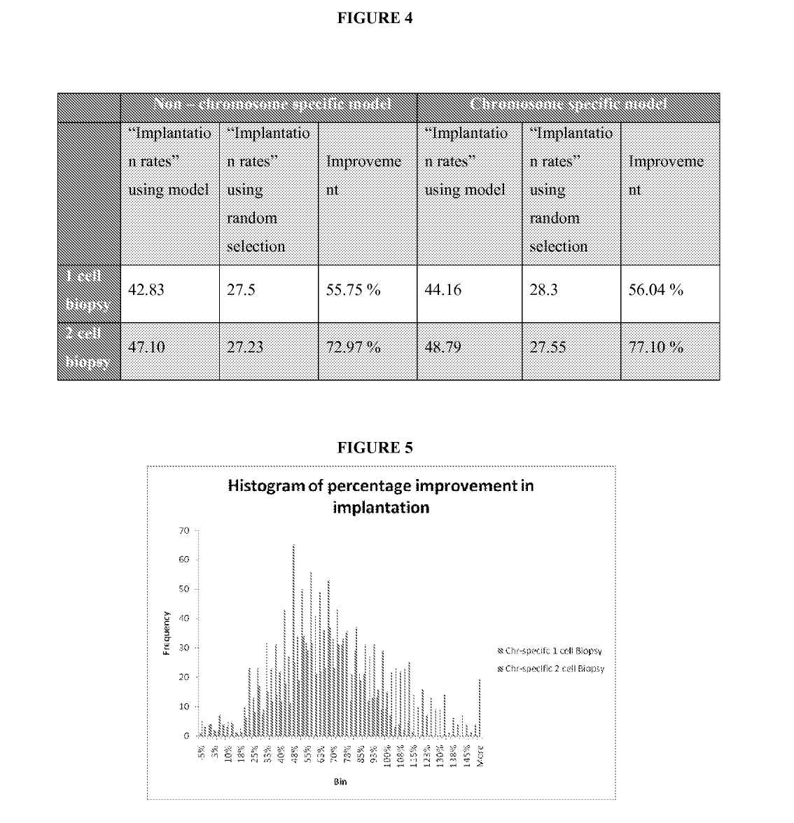

In an embodiment of the present disclosure, the method may be able to differentiate embryos that may have been shown to be aneuploid. Typically, such embryos are either discarded or else they are implanted without regard to the type of aneuploidy detected, except in the exclusion of aneuploidy that can lead to a trisomic birth. In an embodiment, the embryos may be ranked in terms of their relative likelihood to develop as desired. In an embodiment, the embryos may be selected based on the relative likelihood that the embryos may result in a normal birth. One advantage of some embodiments of this method may be to increase in the success rate of IVF cycles where this method is utilized. For example, when this embodiment was applied to an empirical data set, the embryo ranking method resulted in improvements of implantation rates of 50-80% as compared to random selection of aneuploid embryos, such as may be seen in the embodiment of FIG. 4.

### DEFINITIONS

Segment of a Chromosome may mean a section of a chromosome that can range in size from one base pair to the entire chromosome.
Chromosome may refer to either a full chromosome, or a segment of a chromosome.
Genetic data ‘in’, ‘of’, ‘at’ or ‘on’ an individual may all refer to the data describing aspects of the genome of an individual. They may also refer to one or a set of loci, partial or entire sequences, partial or entire chromosomes, or the entire genome.
Ploidy calling, also “chromosome copy number calling”, may be the act of determining the quantity and chromosomal identity of one or more chromosomes present in a cell.
Ploidy State may be the quantity and chromosomal identity of one or more chromosomes in a cell.
Characteristic may refer to any feature that may be used to describe or define an embryo. A characteristic may be a physical characteristic, or it may be genetic in nature. It may refer to any feature of a nucleic acid sequence, including the presence or absence of one or more nucleic acid bases ranging from a SNP to an entire chromosome. Each characteristic may contain one or more subcharacteristics, e.g. ploidy state may be aneuploid, mosaic or euploid; aneuploidy may be further described or defined as nullsomy, monosomy, disomy, trisomy or tetrasomy; trisomy may be UCA or MCA; a genetic sequence may be made up of a plurality of genes; a gene may contain a plurality of single nucleotide polymorphisms (SNPs). Some examples of a characteristic may include: a genetic sequence, a SNP, a point mutation, an insertion, a deletion, the ploidy state, the parental origin of a chromosome, a type of aneuploidy, and poor morphology. While certain embodiments distinguish between characteristic and subcharacteristic as described above, some embodiments of the present disclosure may use the two terms interchangeably and in particular, may use the term characteristic to mean a subcharacteristic.
Physical characteristic may refer to a physical feature, as opposed to a genetic feature. The physical features that may be observed under a microscope include, for example, morphology, size, shape or color. An example of an undesired physical characteristic is poor embryo morphology, typified by, among other things, low proximity of the pronuclei, poor centering of the pronuclei, and/or polarization of the nucleolar precursor bodies.
Genetic condition may refer to any characteristic, or set of characteristics that are genetic in nature. It may refer to a characteristic that is indicative of a phenotype. The phenotype may be a disease. The genetic condition may necessarily imply the presence of a phenotype, or it may imply an increase or decrease in the likelihood that a phenotype will occur. The phenotype may be desired or undesired. Some examples of desired phenotypes may be high intelligence, low cholesterol, and high physical endurance. Some examples of undesired phenotypes may be predisposition toward autism, cystic fibrosis, muscular dystrophy, Down syndrome, Cri du chat syndrome, predisposition toward psoriasis, increased likelihood of breast cancer, low intelligence, predisposition toward heart disease and fragile X syndrome.
Characterizing may refer to analyzing a set of embryos by determining one or more characteristics or subcharacteristics. The determination of one or more subcharacteristics of one or more cells may be used to determine the predicted probability of an embryo containing those cells developing as desired. The analysis may involve grouping the embryos based on one or more characteristics and/or subcharacteristics. It may involve labeling the groups based on the one or more characteristics and/or subcharacteristics. Examples of characteristics and subcharacteristics useful to characterize an embryo may include: aneuploid, euploid, mosaic, MCA trisomy, UCA trisomy, maternal MCA trisomy, monosomy, paternal monosomy, tetrasomy, and physical characteristics. Characterizing an embryo may involve a relative characterization, for example, the embryos or groups of embryos may be labeled good, okay, bad, 1^{st}, 2^{nd}, 3^{rd}, 4^{th}, best, second best, third best, and/or least desirable.
Develop as desired, also ‘develop normally,’ may refer to a viable embryo capable of implanting in a uterus and resulting in a pregnancy. It may also refer to the pregnancy continuing and resulting in a live birth. It may also refer to the born child being free of chromosomal abnormalities. It may also refer to the born child being free of other undesired genetic conditions such as disease-linked genes. The term develop as desired encompasses anything that may be desired by potential parents or healthcare facilitators. In some embodiments, “develop as desired” may refer to an unviable embryo that is useful for medical research or other purposes or may refer to an embryo with a genetic condition, such as downs syndrome, which may be considered undesirable by some parents, but to the decision makers for this embryo (e.g., parents or healthcare providers), this genetic condition is desired.
Chromosomal identity may refer to the referent chromosome number. Normal humans have 22 types of numbered autosomal chromosomes and two types of sex chromosomes. It may also refer to the parental origin of the chromosome. It may also refer to the genetic sequence of the chromosome. It may also refer to other identifying features of a chromosome.
Insertion into a uterus may refer to the process of transferring an embryo into the uterine cavity in the context of in vitro fertilization or any other way or means of allowing an embryo to mature, including a human or animal uterus, a man-made uterus-like environment or a lab.
Group may refer to a set of zero, one, two or more embryos that share at least one characteristic. Groups may be defined by one or more specific characteristics or subcharacteristics. If no embryos fall within a predefined group, then the group will have zero embryos. In some instances particular groups may contain only one embryo.
Disease-linked gene may refer to one or a set of genetic variations, including substitutions, insertions, deletions, or other mutations, that are correlated with a disease. Some examples of disease-linked genes include ΔF508 on the CFTR gene on chromosome 7, which is linked to cystic fibrosis, BRCA2 on chromosome 13, which is linked to breast cancer, or PBX1 on chromosome 9, which is linked to heart disease. In some embodiments, the term “disease-linked gene” may refer to presently known genes or gene markers which indicate a propensity or probability that a particular disease may develop, and genes/gene markers that are determined after the filing of the present application.
Informatics based method may refer to a method designed to determine the ploidy state or the genotype at one or more alleles by statistically inferring the most likely state, rather than by directly physically measuring the state.
Aneuploidy may refer to the state where the wrong number of chromosomes are present in a cell. In the case of a somatic human cell it may refer to the case where a cell does not contain 22 pairs of autosomal chromosomes and one pair of sex chromosomes. In the case of a human gamete, it may refer to the case where a cell does not contain one of each of the 23 chromosomes. When referring to a single chromosome, it may refer to the case where more or less than two homologous chromosomes are present.
Matched copy error, also ‘matching chromosome aneuploidy’, or ‘MCA’ may be a state of aneuploidy where one cell contains two identical chromosomes. This type of aneuploidy may arise during the formation of the gametes in mitosis, and may be referred to as a mitotic non-disjunction error.
Unmatched copy error, also “Unique Chromosome Aneuploidy” or “UCA” may be a state of aneuploidy where one cell contains two chromosomes that are from the same parent, and that may be homologous but not identical. This type of aneuploidy may arise during meiosis, and may be referred to as a meiotic error.
Embryo ranking may refer to the practice of ordering a set of embryos by their likelihood to implant and develop as desired. It may refer to sequentially ordering the embryos. The ranking may be from most likely to develop as desired to least likely to develop as desired. More than one embryo may have the same ranking. It may also refer to the act of selecting one or more embryo(s) that may have the greatest likelihood of developing as desired.
Mosaicism may refer to a set of cells in an embryo, or other being, that are heterogeneous with respect to their ploidy state.
Bins may refer to one or more groups into which each embryo, or chromosome is categorized.
Parental Contexts may refer to the genetic state of a given SNP, on each of the two relevant chromosomes for each of the two parents. The parental context for a given SNP may consist of four base pairs, two paternal and two maternal; they may be the same or different from one another. It is typically written as “m₁m₂|p₁p₂”, where m₁ and m₂ are the genetic state of the given SNP on the two maternal chromosomes, and the p₁ and p₂ are the genetic state of the given SNP on the two paternal chromosomes. Note that in this disclosure, A and B are often used to generically represent base pair identities; A or B could equally well represent C (cytosine), G (guanine), A (adenine) or T (thymine). Also, in a parental context, such as AA|BB, may be used to refer to the set or subset of all SNPs with that context. For example, if the mother is homozygous, and the father is heterozygous, there are nine possible parental contexts: AA|AA, AA|AB, AA|BB, AB|AA, AB|BB, AB|AB, BB|AA, BB|AB, and BB|BB. Every SNP on a chromosome, excluding the sex chromosomes, has one of these parental contexts. The set of SNPs wherein the parental context for one parent is heterozygous may be referred to as the heterozygous context.
Phasing may refer to the act of determining the haplotypic genetic data of an individual given unordered, diploid genetic data.
Non-Disjunction Error or Disjunction Error may refer to a type of error that may occur during mitosis where the duplicated chromosomes are not separated equally into the two daughter cells, resulting in one or both of the daughter cells having an aneuploid number of chromosomes.
Hypothesis may refer to a possible state being statistically considered. This state may the ploidy state.
Leave one out Training may refer to the process of training an algorithm that involves using a single observation from the original sample as the validation data, and the remaining observations as the training data.
Heterozygosity may refer to the measure of the genetic variation in a population; with respect to a specific locus, stated as the frequency of heterozygotes for that locus.
Homologous Chromosomes may be chromosomes that contain the same set of genes and that may normally pair up during meiosis.
Identical Chromsomes may be chromosomes that contain the same set of genes, and for each gene they have the same set of alleles that are identical.

In any of the above embodiments, more that one cell from each embryo may be used to determine the one or more characteristics or subcharacteristics of the cells in order to estimate the likelihood of the embryo developing as desired. When more than one cell is analyzed, the determining step can be performed on the group of cells from each embryo at a time. Alternatively, the determining step can be performed on single cells from each embryo in parallel or sequence for each more than one cell from each embryo.

In an embodiment, the one or more characteristics may include at least one genetic condition. In an embodiment, the one or more characteristics may include at least one physical characteristic. In an embodiment, the determination of a genetic condition may be done using an informatics based method, such as PARENTAL SUPPORT™. In an embodiment, the at least one genetic condition may include the determination of the ploidy state of the one or more cells. In this embodiment, the ploidy state may be initially determined to be euploid or aneuploid. In an embodiment, the one or more characteristic may include the determination of the subcharacteristic or type of aneuploidy found in the one or more cells. In any embodiment, the one or more characteristics may include at least one of: (i) ploidy state; (ii) any trisomies being UCA or MCA; (iii) parental origin of any aneuploidy; (iv) a physical characteristic of an embryo; (v) a presence or absence of a disease-linked gene; (vi) a count of any aneuploid chromosomes; (vii) a chromosomal identity of any aneuploid chromosomes; (viii) any other genetic condition not listed above.

Some examples of the types of aneuploidy criteria described herein that may be used to group or rank embryos include: maternal vs. paternal trisomies, matching vs unmatching copy errors, the number of chromosomes that are aneuploid, and/or the identity of the aneuploid chromosome(s). Empirical information indicates that embryos with maternal trisomies are less likely to develop properly, and that cells with aneuploidy at certain chromosomes are more likely to develop as desired. In addition, embryos with more chromosomes that test positive for aneuploidy are less likely to develop as desired. Theoretical explanations may account for the tendency of embryos with matching copy errors being more likely to develop as desired than those with unmatching copy errors.

In an embodiment, embryos displaying certain criteria may be excluded from possible insertion into a uterus a priori due to the detection, in at least one of the one or more cells from the embryo(s) to be excluded, of at least one of: (i) a viable trisomy; (ii) a viable uniparental disomy (UPD); (iii) an undesired disease-linked gene; and (iv) poor physical characteristics of an embryo. In an embodiment, any characteristic that would result in an embryo not developing “as desired” can be used to exclude an embryo from further grouping, ranking or further characterization. In an embodiment, any chromosomal abnormality may be used to exclude an embryo from possible insertion into a uterus.

Some embodiments may be used in combination with the PARENTAL SUPPORT™ (PS) method, embodiments of which are described in U.S. patent application Ser. No. 11/603,406 and U.S. patent application Ser. No. 12/076,348, which are incorporated herein by reference in their entirety. In some embodiments, The PARENTAL SUPPORT™ method is a collection of methods that may be used to determine the genetic data, with high accuracy, of one or a small number of cells, specifically to determine disease-related alleles, other alleles of interest, and/or the ploidy state of the cell(s). PARENTAL SUPPORT™ may refer to any of these methods.

The PARENTAL SUPPORT™ method makes use of known parental genetic data, i.e. haplotypic and/or diploid genetic data of the mother and/or the father, together with the knowledge of the mechanism of meiosis and the imperfect measurement of the target DNA, in order to reconstruct, in silico, the genotype at a plurality of alleles, the ploidy state of an embryo or of any target cell(s), and the target DNA at the location of key loci with a high degree of confidence. The PARENTAL SUPPORT™ method can reconstruct not only single-nucleotide polymorphisms (SNPs) that were measured poorly, but also insertions and deletions, and SNPs or whole regions of DNA that were not measured at all. Furthermore, the PARENTAL SUPPORT™ method can both measure multiple disease-linked loci as well as screen for aneuploidy, from a single cell. In an embodiment, the PARENTAL SUPPORT™ method may be used to characterize one or more cells from embryos biopsied during an IVF cycle to determine the genetic condition of the one or more cells.

The PARENTAL SUPPORT™ method allows the cleaning of noisy genetic data. This may be done by inferring the correct genetic alleles in the target genome (embryo) using the genotype of related individuals (parents) as a reference. PARENTAL SUPPORT™ is most relevant where only a small quantity of genetic material is available (e.g. PGD) and where direct measurements of the genotypes are inherently noisy due to the limiting amounts of starting material. The PARENTAL SUPPORT™ method is able to reconstruct highly accurate ordered diploid allele sequences on the embryo, together with copy number of chromosomes segments, even though the conventional, unordered diploid measurements may be characterized by high rates of allele dropouts, drop-ins, variable amplification biases and other errors. The method may employ both an underlying genetic model and an underlying model of measurement error. The genetic model may determine both allele probabilities at each SNP and crossover probabilities between SNPs. Allele probabilities may be modeled at each SNP based on data obtained from the parents and model crossover probabilities between SNPs based on data obtained from the HapMap database, as developed by the International HapMap Project. Given the proper underlying genetic model and measurement error model, maximum a posteriori (MAP) estimation may be used, with modifications for computationally efficiency, to estimate the correct, ordered allele values at each SNP in the embryo.

One part of the PARENTAL SUPPORT™ technology is a chromosome copy number calling algorithm that in some embodiments uses parental genotype contexts. To call chromosome copy number, the algorithm uses the phenomenon of locus dropout (LDO) combined with distributions of expected embryonic genotypes. During whole genome amplification, LDO necessarily occurs. LDO rate is concordant with the copy number of the genetic material from which it is derived, i.e., fewer chromosome copies result in higher LDO, and vice versa. As such, it follows that loci with certain contexts of parental genotypes behave in a characteristic fashion in the embryo, related to the probability of allelic contributions to the embryo. For example, if both parents have homozygous BB states, then the embryo will never have AB or AA states. In this case, measurements on the A detection channel will have a distribution determined by background noise and various interference signals, but no valid genotypes. Conversely, if both parents have homozygous AA states, then the embryo will never have AB or BB states, and measurements on the A channel will have the maximum intensity possible given the rate of LDO in a particular whole genome amplification. When the underlying copy number state of the embryo differs from disomy, loci corresponding to the specific parental contexts behave in a predictable fashion, based on the additional allelic content that is contributed or is missing from one of the parents. This allows the ploidy state at each chromosome, or chromosome segment, to be determined

### A Model for the Creation of Mosaicism:

In an embodiment, the present disclosure may be used to enable a clinician, or other agent, to identify one or more embryos, from among a set of embryos, that are the most likely to develop as desired. Typically, embryos that test negative for chromosomal abnormalities, such as aneuploidy, may be chosen for transfer. However, in some cases, there may be insufficient or no embryos that test negative for chromosomal abnormalities such as aneuploidy. In this case, embryos from which one cell has tested positive for a chromosomal abnormality may be aneuploid, or they may be mosaic. Mosaic cells may self correct, and have the potential to implant and develop as desired. In an embodiment, the present disclosure may be used to determine which embryo(s) are most likely to develop as desired. In an embodiment, the grouping or relative ranking of embryos may be made based on a model of mosaicism and how is arises during the development of the embryo.

Within an embryo, different distributions of cells of different ploidy states may occur, and embryos with some of those distributions are more likely than others to develop as desired. An embodiment may utilize the measured genetic condition in one cell from one or more embryo to predict the likely genetic condition in the remaining cells in the embryo. In this embodiment, the genetic condition may be the ploidy state. This measurement may be used to determine whether the cells of an embryo are likely to be euploid, aneuploid, or mosaic, and hence the relative likelihood of that embryo to develop as desired.

In an embodiment of the present disclosure, the present method may assume that the rates of aneuploidy and mosaicism may tend to increase as an embryo develops from the 2 cell to the 8 cell stage. This embodiment may also assume that aneuploidy in embryos often may be accompanied by mosaicism. In an embodiment, the above assumptions may be used to determine the distribution of aneuploidy states in one or more cells from an embryo. In an embodiment, the method may also assume that mosaicism is caused predominantly by errors in mitotic disjunction during embryo growth.

For example, consider that each chromosome has a probability of a non-disjunction error during mitosis. Each time a disjunction error occurs during the mitosis of a cell that is euploid at a given chromosome, that chromosome will have 0 copies of that chromosome in one of the post-division cells and 2 identical copies of that chromosome in the other post-division cell; therefore, both of these post-division cells are now aneuploid. If no error occurs, a chromosome will have 1 copy of each of the identical chromosomes in each of the two post-division cells. Further divisions of such an aneuploid cell will result in daughter aneuploid cells, with the exception of the unlikely event that a non-disjunction error occurs during the division of a cell that is trisomic at a chromosome that results in one of the duplicated identical chromosomes not being passed on to the daughter cell.

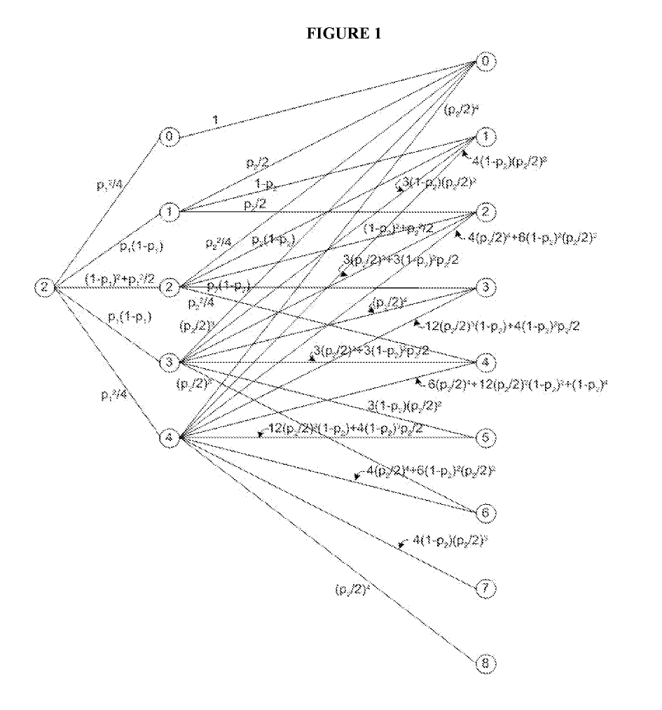

FIG. 1 is a graphical illustration of how, after two divisions, there will be a distribution of probabilities on each of the possible copy numbers of a particular chromosome in a cell. The number of copies of the chromosome is shown in the circles, and the lines between circles represent the transition probability of going from some number of chromosomes to the other during a division. The circle on the left represents a euploid parent cell. The column of circles in the middle represent the possible ploidy states of that chromosome after one division, and the column of circles on the right represent the possible ploidy states after two divisions. One may assume that the probability of a non-disjunction error is the same for each chromosome and that the probability is independent of the number of chromosomes in the pre-division cell. For the first division, the probability of a non-disjunction error is p₁ and for the second division the probability is p₂.

The ploidy state of a cell may be measured using the assumption that most errors occur during the first two cell divisions for a series of cells on day 3 embryos. The resulting measurements can be matched with the results of the model in order to estimate p₁ and p₂. Using the transition probabilities illustrated in FIG. 1, it may be possible to compute the probability of each of the possible ploidy states for that chromosome (1 through 8) in terms of p₁ and p₂. Each of these possible states may be considered hypotheses. In one embodiment, these computed probabilities may be compared with the empirical probabilities on each of the measured chromosome numbers in order to solve for p₁ and p₂ that most closely fit the data under a maximum-likelihood algorithm.

One relevant parameter from this analysis is r₁₂=p₁/p₂, describing the ratio of the probabilities of a mitotic disjunction error in the first and second division. If r₁₂ is close to 1, the distinction between p₁ and p₂ may be eliminated and the disjunction error at each division can be characterized simply as p. This model may be extended to incorporate errors at the third division (the probability of which is indicated by p₃). The model in FIG. 1 may be extended to a third or later division by algebraic methods, or by automated computer simulation, for example using a Monte Carlo method. In one embodiment of the present disclosure, this method may be used to calculate the likelihood of various ploidy states by modeling potential disjunction errors over fewer than two divisions. In an embodiment, this method may be used to calculate the likelihood of various ploidy states by modeling potential disjunction errors over two divisions. In one embodiment, the method can be used to calculate the likelihood of various ploidy states by modeling disjunction errors over three divisions. In another embodiment, the method can be used to calculate the likelihood of various ploidy states by modeling disjunction errors over four, five, six, seven or more divisions.

For the purpose of explanation, one may assume that the first division represents the first mitotic division after the completion of Meiosis II and the extrusion of the polar body following fertilization of an egg by a sperm. Disjunction errors that affect the formation of the sperm or the egg will tend to give rise to cells with additional chromosomes that do not exactly match other chromosomes because crossovers were involved in their formation which are different to the crossovers that gave rise to the other chromosomes in the post-division cell. However, disjunction errors in the divisions illustrated in FIG. 1 will give rise to cells with chromosomes that are exact copies of other chromosomes in the post-division cell. These are referred to as matching chromosomes aneuploidies, or MCAs. If the error occurs before the divisions in FIG. 1, either affecting the sperm or the egg or the fertilized egg, then it is likely that this would cause a unique chromosome aneuploidy, or a UCA.

In one embodiment of the present disclosure, a mechanism that may be used to explain mosaicism in embryos is used, together with the determination of one or more characteristics or subcharacteristics made on one or more cells, in order to determine one or more characteristic or subcharacteristics of other, untested cells within the embryo. If the egg or sperm is affected by an aneuploidy, then it is likely that all blastomeres in the embryo will be affected. Hence, if a UCA is measured, then the embryo has a relatively low probability of having any normal cells; if an MCA is measured, then there is a relatively high probability that the embryo contains some normal cells. In one embodiment of the present disclosure, the one or more characteristics may include the genetic condition of the one or more cells. In one embodiment, the one or more characteristics may include the ploidy state of one or more cells. In one embodiment of the present disclosure, a method, such as PARENTAL SUPPORT™, may be used to determine the subcharacteristics of the one or more cells, such as the type of aneuploidy in a cell.

An embodiment of the present disclosure may include a method of characterizing an embryo for insertion into a uterus, including: selecting at least one characteristic; determining a first subcharacteristic of the at least one characteristic of at least one cell from an embryo; using the determined first subcharacteristic, predicting a probability of a second cell from the embryo having a second subcharacteristic; and characterizing the embryo based on the predicted probability. In an embodiment of the present disclosure, the determination step is performed on more than one cell from an embryo. In an embodiment of the method, the predicting step encompasses using the first subcharacteristic determined to predict probabilities of a plurality of cells from the embryo having a plurality of subcharacteristics. In an embodiment of the present disclosure, characterizing an embryo includes characterizing the embryo based on all of the predicted probabilities associated with each determined subcharacteristic. An embodiment of the present disclosure further includes repeating the determining, predicting and characterizing steps for a plurality of embryos. In an embodiment, the determining step includes using an informatics based method to determine the first subcharacteristic, such as the PARENTAL SUPPORT™ method.

In an embodiment, the at least one characteristic may include at least one genetic condition. In an embodiment, the at least one characteristic may include a ploidy state. In an embodiment, the first subcharacteristic may be one of euploid or aneuploid. In an embodiment, the at least one characteristic includes at least one of: (i) a ploidy state; (ii) any trisomies being UCA or MCA; (iii) parental origin of any aneuploidy; (iv) a presence or absence of a disease linked gene; (v) a count of any aneuploid chromosomes; (vi) a chromosomal identity of any aneuploid chromosomes; (vii) any other genetic condition; and (viii) a type of aneuploidy. In an embodiment, the first at least one characteristic is defined by one or more subcharacteristics.

In one embodiment of the present disclosure, the characterizing step includes grouping the embryo into a group defined by at least one subcharacteristic, wherein each group contains zero, one or more embryos, and any embryos within a particular group share at least one characteristic. In an embodiment of the present disclosure, the characterizing step includes ranking the embryo based on an estimated likelihood of that embryo developing as desired. In an embodiment of the present disclosure, the ranking of embryos is performed to select at least one embryo to insert into a uterus. In an embodiment, a Monte-Carlo simulation is used to predict the probability of the second cell.

In an embodiment of the present disclosure, subcharacteristics may include at least one of (i) aneuploid, mosaic or euploid; (ii) UCA trisomy or MCA trisomy; (iii) maternal or paternal; (iv) present or absent; (v) one, two, three, four, five, six, seven, eight, nine, ten, eleven, twelve, thirteen, fourteen, fifteen, sixteen, seventeen, eighteen, nineteen, twenty, twenty-one, twenty-two, twenty-three or twenty-four; (vi) chromosome one, chromosome two, chromosome three, chromosome four, chromosome five, chromosome six, chromosome seven, chromosome eight, chromosome nine, chromosome ten, chromosome eleven, chromosome twelve, chromosome thirteen, chromosome fourteen, chromosome fifteen, chromosome sixteen, chromosome seventeen, chromosome eighteen, chromosome nineteen, chromosome twenty, chromosome twenty-one, chromosome twenty-two, X chromosome or Y chromosome; (vii) Aneuploidy, Breast cancer (BRCA1), Congenital Adrenal Hyperplasia, Cystic Fibrosis, Duchenne Muscular Dystrophy, Familial Adenomatous polyposis coli (FAP), Familial Alzheimer's disease, Fragile X, Hemophilia, Huntingtons Disease, Klienfelters Syndrome, Marfans Syndrome, Myotonic Dystrophy, Sickle Cell Disease, Spinal Muscular Dystrophy, Tay Sach's Disease, Thalassemia, Translocation, Wiskott-Aldrich syndrome or X-Linked Mental Retardation; and (viii) nullsomy, monosomy, disomy, trisomy or tetrasomy.

In an embodiment, an embryo may be excluded a priori from consideration of insertion into a uterus due to a prediction, in at least one cell of the embryo, of at least one of: (i) a viable trisomy; (ii) a viable uniparental disomy; (iii) any other chromosomal abnormality; (iv) an undesired disease linked gene; and (v) poor physical characteristics of an embryo.

Embryos that are euploid are typically considered most likely to develop as desired; embryos that are mosaic may be considered less likely to develop as desired, and embryos that are aneuploid may be considered the least likely to develop as desired. An embodiment may use the determined ploidy state of one or more cells from an embryo, along with a model of how mosaicism arises, to determine the likely ploidy states of the untested cells in an embryo. In this embodiment, the determined ploidy state of the measured cells may be used to predict the fraction of remaining, untested cells that are euploid, and therefore the likelihood that a given embryo will develop as desired if transferred to a receptive uterus. Another embodiment of the present disclosure may use the determined ploidy state of one or more cells from an embryo, in combination with empirical embryo development data to predict the probability of the ploidy state of the untested cells. In the above embodiments, the information generated above on the tested and untested cells may be used to determine the likelihood that a given embryo will develop as desired if transferred to a receptive uterus.

### Calculating Aneuploidy Type Probabilities

In one embodiment of the present disclosure, the type of aneuploidy measured in cell(s) taken from an embryo may be used to determine the relative likelihood that some or all of the remaining cells in the embryo are euploid. This determination may be based in part on the fact that UCAs are indicative of meiotic errors and MCAs are indicative of mitotic errors, and that embryos containing cells with meiotic stage errors are less likely to contain euploid cells than embryos that have one or more cell with a mitotic state error. Additionally, in some embodiments, it may be assumed that embryos completely made up of aneuploid cells are less likely to develop as desired than those containing euploid or mosaic cells. Given the nature of the various disjunction errors, it may be assumed that embryos with measured UCAs are less likely to develop as desired than embryos with measured MCAs. In the case of uniparental disomies (UPD) and tetrasomies it is possible to conduct a similar analysis to determine whether the observed aneuploidy is more likely due to a meiotic error or whether the observed aneuploidy is due to a mitotic error. In an embodiment of the present disclosure, it may be assumed that the chance of euploid cells after a mitotic error has occurred is greater than the chance of euploid cells after a meiotic error has occurred.

In one embodiment of the present disclosure, the probability that a given embryo that tested aneuploid at one or more cells may contain some euploid cells may be calculated. The probability that an untested cell taken from the an embryo in which one or more cells is tested is euploid is designated P(E). In an embodiment, P(E) may be estimated using the probability of each of the trajectories tᵢ, i=1 . . . T in FIG. 1 that could have given rise to the measured copy number on each chromosome.

In one embodiment of the present disclosure, the present method may be used to estimate of the probability P(E) for one chromosome at a time. In order to estimate this probability, P(E) may be calculated as follows:

$$P ( E \vert M ) = {\sum }_{i = 1 … T} P ( E \vert {t}_{i} ) P ( {t}_{i} \vert M )$$

wherein M is the measurement of a chromosome copy number, P(E|tᵢ) is the probability that another cell in the embryo is diploid on the chromosome of interest, given trajectory tᵢ and P(tᵢ|M) denotes the probability of the trajectory tᵢ given the measurement M. This may be computed as follows:

$$P ( {t}_{i} \vert M ) = \frac{P ( M \vert {t}_{i} ) P ( {t}_{i} )}{P ( M )}$$

P(M|tᵢ)=1 if tᵢ is a trajectory that results in that measured number of chromosomes, M, and 0 otherwise. Hence, for relevant trajectories, it may be assumed that P(tᵢ|M)=P(tᵢ)/P(M), which can be computed from FIG. 1 by looking at the probability of trajectory tᵢ over all possible trajectories that give rise to measurement M.

$$P ( {t}_{i} \vert M ) = \frac{P ( {t}_{i} )}{{\sum }_{i s . t . trajectory ti generates M} P ( {t}_{i} )}$$

In one embodiment, the probability that another cell in the embryo is disomic at that chromosome P(E|tᵢ), may be computed, given that the biopsied cells followed trajectory tᵢ. This may be computed either in closed form or by a method such as a Monte-Carlo method where the replication and division of the chromosomes from one cell to the 8 cell stage is simulated. In one embodiment, it may be assumed that one cell is forced to follow trajectory tᵢ, and P(E|tᵢ) may be calculated by simply counting the number of other cells that are euploid on that chromosome over many simulations. In an embodiment, other mathematical or computer based methods may be used as applicable, and any number of divisions may be modeled. In an embodiment, two or three divisions may be modeled. In an embodiment, four, five, six, seven or more divisions may be modeled.

In an embodiment of the present disclosure, a method is given here to estimate P(E_{c}), for multiple chromosomes, where P(E_{c}) denotes the probability that a cell in the embryo is euploid on chromosome c, c=1 . . . 24. This embodiment may use the method for estimating P(E) for an individual chromosome, described above, and repeating it for all chromosomes. In an embodiment, one may compute P(E_{c}|M) to rank the embryos. Assuming that all chromosomes are independent, one may estimate the probability that a particular embryonic cell is euploid in all chromosomes as:

P(euploid on all chromosomes)=Π_{c}P(E_{c}|M_{c})

In one embodiment of the present disclosure, P(E_{c}) may be calculated as above for a subset of the 24 chromosomes by simply taking c to be the desired number between 2 and 23. In another embodiment of the present disclosure the expected number of euploid cells in an embryo may be computed with a set number, N, of cells before biopsy as follows:

Expected Euploid Cells=(N−1)P(euploid on all chromosomes).

In another embodiment of the present disclosure, the probability that another cell taken from the same embryo is euploid may be calculated, P(E), after the biopsy and analysis of a plurality of blastomeres.

To do this, let M_{c1} and M_{c2} represent the measurement on chromosome c in cells 1 and 2. In one embodiment, P(E|M_{c1}, M_{c2}) may be calculated in closed form. In one embodiment, P(E|M_{c1},M_{c2}) may be computed by Monte-Carlo simulation of the model. In one embodiment, P(E|M_{c1}, M_{c2}) may be calculated by simulating multiple three stage divisions as above, and for all cases that result in two cells with respective measurements M_{c1} and M_{c2}, find the fraction of the other cells in the embryo with a disomic chromosome c.

In another embodiment of the present disclosure, the probabilities p₁, p₂ and p₃, i.e., the scenario in which three mitotic events occur, may be calculated on a per-sample basis rather than aggregated over multiple samples. In this embodiment, the ratios r₁₂=p₁/p₂ and r₂₃=p₂/p₃ may be calculated from the aggregated data, as described above, using the assumption that this ratio stays roughly the same from one sample to another. This embodiment may use the estimate p₁ for each sample, which may be simplified as p, and M denotes the set of measurements on all chromosomes in a cell: M={M_{c}}, c=1 . . . 24. In this embodiment, p may be calculated using maximum a posteriori probability and Bayes Rule:

$$p = arg {max}_{p} P ( p \vert M ) = arg {max}_{p} \frac{P ( M \vert p ) P ( p )}{P ( M )}$$

In some embodiments, it is possible to maximize over p one may drop the denominator P(M), and P(p) may be computed from the aggregated data over multiple embryos. In an embodiment, each of the measurements M_{c} may be treated as conditionally independent given p, hence we find p from:

p=arg maxₚΠ_{c}P(M_{c}|p)P(p)

where P(M_{c}|p) is straightforward to compute based on simulation or in closed form from FIG. 1. This embodiment, may be extended to the two cell biopsy case, in which the ploidy state may be measured on all chromosomes on both cells M={M_{1,c},M_{2,c}}, c=1 . . . 24 and the determination of p may be written as:

p=arg maxₚΠ_{c}P(M_{1,c},M_{2,c}|p)P(p)

where P(M_{1,c},M_{2,c}|p) may be found by simulation. In this embodiment, the resultant value of p may be used to compute P(euploid on all chromosomes) as described above, which may be then used to rank embryos.

In another embodiment, one could use a similar approach to compute the probability that at least one cell is euploid at that chromosome. In an embodiment, the above calculations may be used to determine whether at least 25%, at least 50%, at least 75% or at least 100% of the cells are euploid at that chromosome. In an embodiment, P(N_{ec}|M_{c}) may also be directly estimated by Monte-Carlo, or other computer based simulation, rather than breaking it down into the constituent terms.

In an embodiment, the probability calculations above account for the assumption that the number of cells with various types of aneuploidy in a cell may change as the embryo develops, and the probability that an embryo will develop as desired may depend partly on the number and ploidy state of those cells.

In one embodiment of the present disclosure, cells with aneuploidy on a preselected set of chromosomes, for example trisomy 8, 13, 21, X and/or Y, may be eliminated from consideration for implantation a priori. In another embodiment, other sets of ploidy states on other chromosomes may be used for a priori selection.

In another embodiment, a model of mosaicism which allows for chromosomes to be lost may be used. In the embodiment described above, an assumption is made that the two post-division cells contain, between them, both of the copies of a chromosome that divides during mitosis, either equally (1,1) or in an imbalanced fashion due to mitotic non-disjunction (0,2) or (2,0). In this embodiment, a model may be used that allows for the possibility that a chromosome is completely lost during disjunction so that the state of the chromosome in the post-division may be any of the following: 1,1 or 0,2 or 2,0 or 1,0 or 0,1 or 0,0. In another embodiment of the present disclosure, a model may be used that assumes that other possibilities may occur upon cell division, such as extra copies of a chromosome being produced.

In an embodiment, data from Hapmap or similar data concerning crossover likelihoods during meiosis, may be used to determine the probability that a non-disjunction error occurred during meiosis to give rise to a UCA or an MCA. In this embodiment, an informatics based approach, such as PARENTAL SUPPORT™, may be used to take advantage of crossover probabilities, and may phase the genetic data of the blastomere. In this embodiment, one may identify chromosomes that have matching crossovers, or other characteristics that indicate that the non-disjunction error occurred during meiosis, and make that determination for each chromosome.

### Embryo Ranking

The general concept behind embryo ranking is to categorize embryos into groups or bins that have different probabilities of developing normally, and then to rank the embryos by those relative probabilities. In one embodiment of the present disclosure, the ranking may be used to decide which embryo(s) to transfer in the context of IVF. In one embodiment, the first step is to differentiate embryos into groups and then calculate the probability that the embryos in each of the bins have to develop as desired. In an embodiment, the relative probability of an embryo to develop as desired may be calculated, using contingency tables, using published embryo development data, using other sources of empirical embryo development data, using a combination of various sources of embryo development data, or using embryo development theories. In an embodiment, those probabilities can then be used to determine which embryo(s) to transfer in the context of IVF; this may be done by selecting the embryo whose calculated probability of developing normally is the greatest. Many of the embodiments described herein focus on methods of differentiating the embryos using bins related to particular ploidy states. Some examples of the types of ploidy states that may be used to categorize the embryos include MCA trisomy and UCA trisomy, the parental origin of any aneuploid chromosomes, the number of aneuploid chromosomes observed, the identity of aneuploid chromosomes, or some combination thereof. The embryos may also be differentiated using other physical characteristics, for example, embryo morphology, embryo size, or the absence or presence of certain genotypes.

In one embodiment, the first step may be to decide on a set of groups, or bins, and a method that may be used to divide the embryos into those groups. Each bin may be defined by a set number of characteristics that are each associated with a probability of normal embryo development. In this embodiment, the next step may be to determine the probabilities that the embryos in each of those groups is likely to develop as desired. In this embodiment, the probabilities may be determined using empirical data and calculating those probabilities, or by other methods described elsewhere in this document.

The number of bins may be very small, for example two, or the number of bins may be very large, such that after categorization, only a small percentage of the bins are populated, or the number may be anywhere in between. Any number of bins may be used. In one embodiment, a large number of bins may be used so that each embryo may be differentiated from every other, and the ranking will be more specific. In some embodiments, some of the bins may have essentially equal probabilities associated with them. In an embodiment, a small number of bins may be used so that the calculation of the likelihood that embryos in a given bin have to develop as desired is based on a limited amount of empirical embryo development data. The fewer the bins, the more empirical data will be available for each bin, and thus the more accurate the prediction may be.

In one embodiment, subcharacteristics, such as basic ploidy states may be used as bins: nullsomy, monosomy, disomy and trisomy. In another embodiment, the trisomic bin may be separated into MCA trisomies, and UCA trisomies. In another embodiment, each chromosome may be considered separately, so that, for example, if each chromosome is categorized into five bins, then 5²³ bins would be used. Some bins may contain no embryos. In some embodiments the bins may reflect the possibility of the ploidy state being known for more than one cells from an embryo, and that those ploidy determinations may or may not correspond. In some embodiments, two or three bins may be used. In some embodiments five to ten bins may be used. In some embodiments, ten to one hundred bins may be used. In some embodiments, one hundred to one million bins may be used. In another embodiment, one could train more fine grained probabilities than just the P(D/t), P(D/m), P(D/n). In one embodiment, embryos may be ranked based on more complex abnormalities, for example, a combination of a monosomy and a trisomy, or two trisomies.

### Distinguishing Meiosis I/II Errors and Mitosis Errors

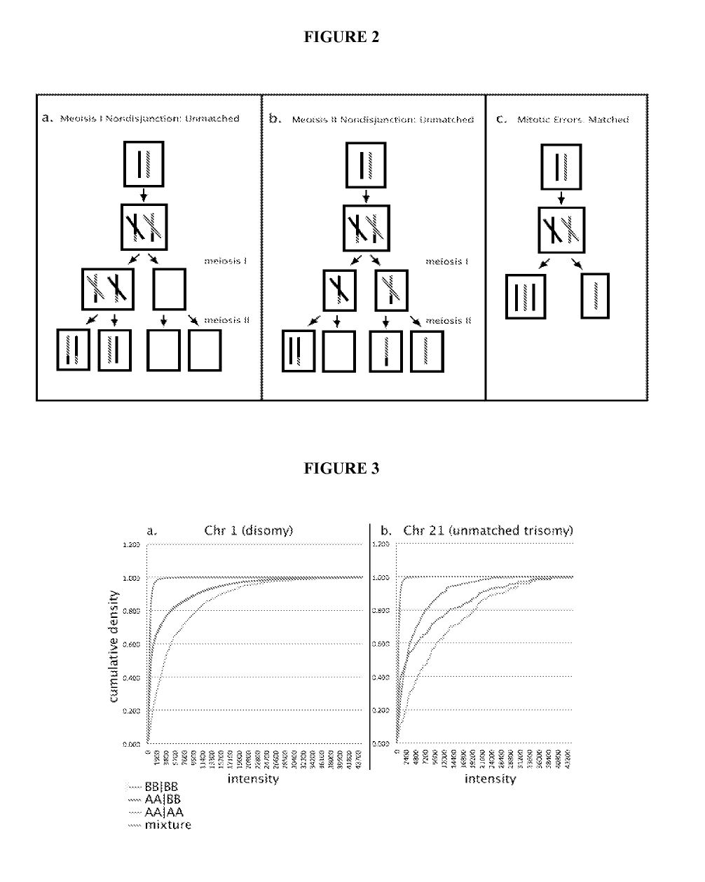

In one embodiment of the present disclosure, the embryos may be differentiated by the type of non-disjunction error. For example, they may be differentiated by errors that most likely occurred during meiosis, and those that likely occurred during mitosis. Matched errors, (MCA) where two of the three chromosomes of a trisomy are identical, will generally indicate mitotic errors; unmatched errors, (UCA) where all three homologues of a trisomic pair of chromosomes are different, will generally indicate that recombination likely occurred in meiosis I between homologous chromosomes to create a tetratype chromosome state. This concept is illustrated in FIG. 2. In an embodiment, the method illustrated in FIG. 2 may be used to determine the type of non-disjunction errors. In an embodiment, other methods may be used to decipher the type of non-disjunction errors. In one embodiment of the present disclosure, one may use a method that uses the parental genotype contexts or parental haplotypes. In one embodiment, a partial or full delineation of parental haplotypes is made, and those haplotypes, along with the measured genetic information from the blastomere, and an informatics method such as PARENTAL SUPPORT™ are used to help determine the ploidy state of the blastomere.

Parental contexts can be highly informative when attempting to determine the embryonic chromosome state. The parental context for a given SNP is the identity of the two corresponding SNPs on both the mother and the father, representing the set of possible SNP identities from which the embryo genotype originates. According to the mechanism of meiosis, in the case of a normal euploid embryo, at a given locus, one SNP will be maternal in origin, and the corresponding SNP on the homologous chromosome will be paternal in origin. The identity of the SNP of maternal origin will be that of one of the two maternal SNPs at that locus, and the identity of the SNP of paternal origin will be that of one of the two paternal SNPs at that locus. The parental context for a given SNP may be written as “m₁m₂|p₁p₂”, where m₁ and m₂ are the genetic state of the given SNP on the two maternal chromosomes, and the p₁ and p₂ are the genetic state of the given SNP on the two paternal chromosomes. The genotype at a given SNP of a euploid embryo with the parental context of m₁m₂|p₁p₂ could be m₁,p₁, m₁,p₂, m₂,p₁ or m₂,p₂.

In one embodiment of the present disclosure, the matched/unmatched discrimination algorithm may use the parental contexts. This embodiment may use a method to determine the difference in the distribution of measured embryonic SNPs between the different parental contexts under matched and unmatched errors. This embodiment is illustrated in FIG. 3. The distribution of measured embryonic SNPs in the heterozygous context is expected to be different for different ploidy states, and when the distributions are considered for all of the contexts, each different embryonic ploidy state has its own characteristic set of distributions. Typically, heterozygosity increases under unmatched errors but stays constant under matched errors.

For example, suppose that loci are randomly selected from the AA|BB and BB|BB contexts on the A microarray detection channel. Under maternal trisomy caused by a MCA, the distribution of AB|BB should look like a bimodal mixture of the loci randomly selected from AA|BB and BB|BB. To illustrate this example, subdivide the A and B contexts each into four subcontexts: A₁ and B₁ are alleles from chromosome copy 1, and A₂ and B₂ are from chromosome copy 2. A matched error consistently results in loci that are A₁B₂B₂|BB and A₂A₂B₁|BB, which results in a context distribution no different than a random selection from A₁A₂|BB, B₁B₂|BB. In contrast, consider the case where the trisomy is caused by a UCA. With an unmatched copy error, there are two more subcontexts, i.e., A₃ and B₃. This results in 3-factorial (six) types of loci in the AB|BB context: A₁B₂B₃|BB, A₁A₂B₃|BB, A₁A₃B₂|BB, A₂A₃B₁|BB, A₃B₁B₂|BB, and A₂B₁B₃|BB. As a result, AB|BB under unmatched trisomy has a trimodal distribution and does not look like a mixture of the distributions of AA|BB and BB|BB. This is because heterozygosity is higher than expected in the case of unmatched trisomy. Thus, to discriminate matched from unmatched errors, one may formulate the null hypothesis as maternal trisomy caused by a matching error, and then attempt to match the cumulative density function of AB|BB with a mixture of the AA|BB and BB|BB cumulative density functions. Established statistical methods such as the Kolmogorov-Smirnov goodness of fit test may be used to determine a confidence interval, and if the difference between the AA|BB/BB|BB mixture and the actual cumulative distribution function (CDF) of AB|BB is in the rejection region, the null hypothesis may be rejected, and it can be concluded that the trisomy is caused by an unmatched error. This may be done separately for both detection channels (X and Y) on Infinium, or other, microarrays, and then the probability of rejection is combined.

Differentiating Meiotic from Mitotic Errors with Phasing (Sperm Genotyping)

In another embodiment of the present disclosure, a method may be used that includes phasing the embryonic data, and determining which chromosomes or segments of chromosomes in the embryo originate from which parent. This method may be particularly useful, for example, in a case where, due to crossover(s) during meiosis, limited exchange of genetic material between homologous chromosomes results in a tetratype where sister chromatids are mostly identical. Although phasing is a challenging problem, methods have been described elsewhere, such as the PARENTAL SUPPORT™ method, that are specifically designed to phase noisy unordered single cell genotype measurements. It is possible to use this capability to differentiate meiotic (UCA) from mitotic (MCA) errors.

In an embodiment of the present disclosure, the present method is used in conjunction with PARENTAL SUPPORT™ and may, assume disomy but may also consider the possibility of trisomy in its theoretical derivation. In this embodiment, for each chromosome, on n SNPs data D=(D₁, . . . , Dₙ) is generated where data on i^{th} SNP consists of (X,Y) channel data for all k blastomeres, 1 sperm cells, mother genomic and father genomic, i.e. Dᵢ=(D^{e}ᵢ,D^{s}ᵢ,D^{m}ᵢ,D^{f}ᵢ), where D^{e}ᵢ=((X^{e}ᵢ₁,Y^{e}ᵢ₁), . . . , (X^{e}ᵢₖ,Y^{e}ᵢₖ)), D^{s}ᵢ=(((X^{s}ᵢ₁, Y^{s}ᵢ₁), . . . , (X^{s}ᵢ₁, Y^{s}ᵢ₁)), D^{m}ᵢ=(X^{m}ᵢ,Y^{m}ᵢ), D^{f}ᵢ(X^{f}ᵢ,Y^{f}ᵢ). In this embodiment, for each embryo target, j=1, . . . , k, on each SNP i, the goal is to derive the most likely allele call g^{j}ᵢ=(n^{A}ᵢⱼ,n^{B}ᵢⱼ), by calculating P(gᵢⱼ|D) for all possible allele values, returning the value with highest probability, and returning that probability as the confidence in that call. In this embodiment, by first calling the copy number classification algorithm, it is possible to derive the copy number hypothesis likelihood given the data P(fⱼ|D,j)=P(copy number hypothesis=fⱼ on jth target|D). For SNP i, on blastomere j:

P(gᵢⱼ|D)=Σ_{F=(f1 . . . fk)}P(gᵢⱼ|F,D)(Π_{t=1 . . . k}P(fₜ|D,t))

where F is the set of copy number hypotheses for all blastomeres. The sum over F=(f₁ . . . fₖ) represents the sum over all possible combinations of hypotheses over all embryo targets 1 . . . k, and P(gᵢⱼ|F,D) is the conditional probability of the allele call assuming a particular set of copy number hypotheses (F) over all blastomeres given the data. It is possible to derive this probability for any value of F, which may include trisomies on particular blastomeres, and to analyze the hypotheses in a set F since the probability of each hypothesis on each blastomere is dependent on the probabilities of the hypotheses on the other blastomeres. If two haplotypes are most likely in a trisomic state, the chromosome may be called matched, and if the hypothesis of three haplotypes is most likely, the chromosomes may be called unmatched. Because the haplotyping method specifically orders the genotype measurements into haplotypes, it may achieve higher sensitivity than some methods.

### Analyzing Polar Bodies and Multiple Single Cells Simultaneously

In another embodiment, polar bodies and/or other cells may be a source of extra information from which embryos can be ranked. In an embodiment, any source of genetic information that correlates with the ploidy state of the embryo can be used, for example, additional cells taken from or originating from the embryo, including polar bodies or any other appropriate source. In an embodiment, the genetic information is gathered from two cells of a 3-day embryo. In another embodiment, the genetic information is gathered from two or more cells from a 5-day embryo. In any of the above embodiments, the additional genetic data is used to validate the prediction of a “normal” embryo based on the scoring scheme. In any of these embodiments, various sets of data can be combined to make increasingly accurate predictions of the actual genetic state of the embryo. In any of these embodiments, the additional genetic information may improve the chance of correctly deducing the ploidy state of the remaining cells in the embryo.

In one embodiment of the present disclosure, the probabilities (e.g. P(D/t1)) may be computed on a per chromosome basis. In another embodiment, this method may be executed on each chromosome segment; that is segment by segment. For example, in a case where low confidences are caused by de novo mitotic translocations, this could be caused by embryos in which one blastomere has a trisomy on a tip and another blastomere has a monosomy on the corresponding tip. This embodiment of the method takes into account unbalanced translocations, and may give more accurate results when said translocations occur at a significant level.

In one embodiment of the present disclosure, the embryos may be grouped based on the parental origin of the chromosomes in the cell. For example, some studies indicate that if a trisomy is detected at a given chromosome on a blastomere, the likelihood that the embryo from which the blastomere was biopsied contains euploid cells is higher if two of the three trisomic chromosomes originate from the father, as opposed to if two of the three trisomic chromosomes originate from the mother. In an embodiment, the parental origin of chromosomes in the case of a uniparental disomy, or a monosomy may be used to categorize the embryos. In this embodiment, if a blastomere is measured to have a paternal monosomy, one would expect an increased likelihood of another cell in the embryo containing a maternal MCA trisomy.

In another embodiment, one may use the number of MCAs in a single cell in order to rank the embryo. In this embodiment, if a cell is determined to have MCAs measured at more than one chromosome, is the embryo would be considered to be less likely to contain euploid cells than an embryo from which one blastomere has been determined to have MCAs measured at only one chromosome. In another embodiment of the present disclosure, different combinations of aneuploidy types at different chromosomes, as measured on a blastomere from that embryo, may be used to categorize the embryos. In another embodiment of the present disclosure, the chromosomal identity of MCAs, or other ploidy states, may be used to rank the embryos. For example, data may show that embryos with an MCA measured at chromosome 3 may be more likely to develop as desired than embryos with an MCA measured at chromosome 6. In another example, a paternal trisomy at chromosome 9 may be considered more likely to develop as desired than a maternal trisomy at chromosome 9. In another example, a monosomy at chromosome 4 may be more likely to develop as desired than a monosomy at chromosome 2.

In another embodiment of the present disclosure, embryos may be differentiated into bins based on properties other than types of aneuploidy. For example, embryos may be differentiated based on the presence or absence of any alleles known to be correlated with implantation and/or the health of a baby. In one embodiment, embryos may be differentiated into bins based on physical characteristics, such as morphology, size, shape, color, transparency, or the presence or absence of various features. In some embodiments of the present disclosure, embryos may be differentiated based on a combination of qualities, such as those listed here. For example, embryos may be differentiated based on ploidy state and morphology; embryos may be differentiated based on ploidy state and the presence of an implantation related alleles; embryos may be ranked based on ploidy state and the parental origin of any trisomies.

In one embodiment of the present disclosure, the embryos are biopsied at day 5 from the tropechtoderm. Trophectoderm biopsy is a newer approach to PGD that assesses the chromosomal status of the trophectoderm immediately prior to implantation. In contrast with single cell biopsies at the 3 day stage, the trophectoderm biopsy typically yields between 4-10 cells. In one embodiment of the present disclosure, the biopsied cells are genotyped together. In this embodiment, the genotyping results may need to be interpreted using non-standard methods. In some embodiments, the tropechtoderm sample may consist of a mosaic population of cells. In this embodiment, the present method may be used in combination with an informatics based methods such as the PARENTAL SUPPORT™ algorithm to choose the optimal hypothesis among a set of hypotheses that describe the various possible states of mosaic aneuploidy in the trophectoderm. In another embodiment of the present disclosure, the individual cells from the tropechtoderm biopsy are separated, and the ploidy state of one or more of them are called individually. In one embodiment, one or two cells may be biopsied from the embryo. In one embodiment, three to ten cells may be biopsied. In one embodiment, eleven to twenty cells may be biopsied. In one embodiment, more than twenty cells may be biopsied. In one embodiment, an unknown number of cells may be biopsied. In one embodiment, the cells may be biopsied at day 2 or day 3. In one embodiment, the cells may be biopsied at day 4, 5 or 6. In one embodiment, the cells may be biopsied later than day 6.

In one aspect of any of the above embodiments, chromosomal abnormalities that give rise to congenital defects may be excluded a priori. Such a congenital disorder may be a malformation, neural tube defect, chromosome abnormality, Down's syndrome (or trisomy 21), Trisomy 18, spina bifida, cleft palate, Tay Sachs disease, sickle cell anemia, thalassemia, cystic fibrosis, Huntington's disease, and/or fragile x syndrome. Chromosome abnormalities include, but are not limited to, Down syndrome (extra chromosome 21), Turner Syndrome (45×0) and Klinefelter's syndrome (a male with 2× chromosomes). In one embodiment, the malformation is a limb malformation. Limb malformations include, but are not limited to, amelia, ectrodactyly, phocomelia, polymelia, polydactyly, syndactyly, polysyndactyly, oligodactyly, brachydactyly, achondroplasia, congenital aplasia or hypoplasia, amniotic band syndrome, and cleidocranial dysostosis. In one aspect of this embodiment, the malformation is a congenital malformation of the heart. Congenital malformations of the heart include, but are not limited to, patent ductus arteriosus, atrial septal defect, ventricular septal defect, and tetralogy of fallot. In another aspect of this embodiment, the malformation is a congenital malformation of the nervous system. Congenital malformations of the nervous system include, but are not limited to, neural tube defects (e.g., spina bifida, meningocele, meningomyelocele, encephalocele and anencephaly), Arnold-Chiari malformation, the Dandy-Walker malformation, hydrocephalus, microencephaly, megencephaly, lissencephaly, polymicrogyria, holoprosencephaly, and agenesis of the corpus callosum. In another aspect of this embodiment, the malformation is a congenital malformation of the gastrointestinal system. Congenital malformations of the gastrointestinal system include, but are not limited to, stenosis, atresia, and imperforate anus.

According to some embodiments, the systems, methods, and techniques of the present disclosure are used in methods to increase the probability of implanting an embryo obtained by in vitro fertilization that is at a reduced risk of carrying a predisposition for a genetic disease. In one aspect of this embodiment, the genetic disease is either monogenic or multigenic. Genetic diseases include, but are not limited to, Bloom Syndrome, Canavan Disease, Cystic fibrosis, Familial Dysautonomia, Riley-Day syndrome, Fanconi Anemia (Group C), Gaucher Disease, Glycogen storage disease 1a, Maple syrup urine disease, Mucolipidosis IV, Niemann-Pick Disease, Tay-Sachs disease, Beta thalessemia, Sickle cell anemia, Alpha thalessemia, Beta thalessemia, Factor XI Deficiency, Friedreich's Ataxia, MCAD, Parkinson disease-juvenile, Connexin26, SMA, Rett syndrome, Phenylketonuria, Becker Muscular Dystrophy, Duchennes Muscular Dystrophy, Fragile X syndrome, Hemophilia A, Alzheimer dementia-early onset, Breast/Ovarian cancer, Colon cancer, Diabetes/MODY, Huntington disease, Myotonic Muscular Dystrophy, Parkinson Disease-early onset, Peutz-Jeghers syndrome, Polycystic Kidney Disease, Torsion Dystonia.

In one embodiment of the present disclosure, the disclosed method is employed in conjunction with other methods, such as PARENTAL SUPPORT™, to determine the genetic state of one or more embryos for the purpose of embryo selection in the context of IVF. This may include the harvesting of eggs from the prospective mother and fertilizing those eggs with sperm from the prospective father to create one or more embryos. It may involve performing embryo biopsy to isolate a blastomere from each of the embryos. It may involve amplifying and genotyping the genetic data from each of the blastomeres. It may include obtaining, amplifying and genotyping a sample of diploid genetic material from each of the parents, as well as one or more individual sperm from the father. It may involve determining the genetic haplotypes of the blastomere, or of the genetic material of related individuals. It may involve incorporating the measured diploid and haploid data of both the mother and the father, along with the measured genetic data of the embryo of interest into a dataset. It may involve using one or more of the statistical methods disclosed in this patent to determine the most likely state of the genetic material in the embryo given the measured or determined genetic data. It may involve the determination of the ploidy state of the embryo of interest using the measured diploid genotype, and an informatics based approach such as PS. It may involve the determination of the ploidy state of the embryo of interest using the distribution of alleles that are detected in a plurality of fractions, each fraction having been created by dividing the genetic material from a single cell prior to amplification and genotyping. It may involve ranking the embryos based on their likelihood to develop as desired and result in the birth of a healthy baby. It may involve the determination of the presence of a plurality of known disease-linked alleles in the genome of the embryo. It may involve making phenotypic predictions about the embryo. It may involve generating a report that is sent to the physician of the couple so that they may make an informed decision about which embryo(s) to transfer to the prospective mother.

It will be recognized by a person of ordinary skill in the art, given the benefit of this disclosure, that various aspects and embodiments of this disclosure may implemented in combination or separately.

### Experimental Section

In one embodiment of the present disclosure, the method was implemented as follows: once the IVF cycle commenced on Day 0 (when harvested eggs had undergone fertilization), the clinic alerted the lab as to the number of fertilized eggs. The embryos underwent morphological evaluation during their development in vitro, and embryos of good morphological quality on Day 3 underwent a single blastomere biopsy for PGD according to standard IVF protocols. The IVF laboratory cultured the embryos to the blastocyst stage using sequential, stage-specific culture media and an advanced, ultra-stable, low-oxygen culture system that is able to adapt to the changing metabolism of the blastulating embryos. The IVF centers then shipped the blastomeres on ice by courier, and the lab received the samples on the morning of Day 4.

Single cells were manually isolated using a micromanipulator (Transferman NK2-Eppendorf). All single cells were washed sequentially in three drops of hypotonic buffer (5.6 mg/ml KCl, 6 mg/ml bovine serum albumin) to reduce the possibility of contamination. Three different lysis/amplification protocols have been used in the analysis: (i) Multiple Displacement Amplification (MDA, GE Healthcare, Piscataway, N.J.) with Alkaline Lysis Buffer (ALB), (ii) Sigma Single Cell Amplification Kit (WGA, Sigma, St. Louis, Mo., USA) with Sigma Proteinase K Buffer (Sigma PKB), (iii) and MDA with Proteinase K Buffer (PKB). In protocol (i) cells were frozen at −20° C. in ALB (200 mM KOH, 50 mM dTT) for 30 minutes, thawed, and neutralized with an acid buffer (900 mM Tris-HCl, pH 8.3, 300 mM KCl, 200 mM HCl). Protocol (ii) was performed according to the manufacturer's instructions. For protocol (iii), cells were placed in PKB (Arcturus PICOPURE Lysis Buffer, 50 mM DTT), incubated at 56° C. for one hour, and then heat inactivated at 95° C. for ten minutes. For protocols (i) and (iii), MDA reactions were incubated at 30° C. for 2.5 hours and then 95° C. for five minutes. Genomic DNA from bulk tissue (Epicentre MASTERAMP Buccal Swabs, Madison, Wis., USA) was isolated using the DNEASY Blood and Tissue Kit (Qiagen, Hilden, Germany). No template controls (hypotonic buffer blanks) were performed for each amplification method.

Both amplified single cells and bulk parental tissue were genotyped using the Illumina (San Diego, Calif., USA) INFINIUM II genome-wide genotyping microarrays (HapMap CNV370DUO or CNV370QUAD chips). For the bulk tissue, the standard Infinium II protocol (www.illumina.com) was used and required call rates of >97% using standard BEADSTUDIO allele calling. Single cells were genotyped using a modified Infinium II genotyping protocol, such that the entire protocol, from single cell lysis through array scanning, was completed in fewer than 24 hours. A variety of time saving modifications were made to the protocol, for example, the duration of the amplification and hybridization steps were reduced by 50% and 63%, respectively. Samples and analytes were tracked using a laboratory information management system (LIMS). Raw data were parsed and used as input for ploidy state analysis.

Upon completing the genotyping assays, the PARENTAL SUPPORT™ method was used to determine the ploidy state of each of the chromosomes in each embryo, including whether any detected trisomies were MCAs or UCAs, and the parental origin of the chromosomes. Each of the 23 chromsomes from the embryos were then categorized into five bins: (1) euploid, (2) one monosomic chromosome, (3) one trisomic chromosome (4) one nullsomic chromosome and (5) other aneuploidy, for a total of 5²³ bins, many of which were statistically treated the same. Embryos whose biopsied blastomere was euploid were considered to be the most likely to implant, and in the cases where euploid embryos were available, those were transferred. A number of aneuploidy states were rejected a priori, these include: trisomy 8, 9, 13, 16, 18, 21, 22 and 23, as well as paternal UPD 6, 11, maternal UPD 7, and any UPD at 14, 15 or 23. Nine embryos that were determined to be aneuploid and were ranked were transferred, along with one euploid embryo, in six IVF cycles. Of those cycles, one pregnancy results. The transferred aneuploid embryos had the following aneuploidy states: (1) monosomy 16, (2) trisomy 16, (3) monosomy 22, (4) monosomy 14, (5) trisomy 15+monosomy 8, 10, 22, (6) monosomy 19, (7) monosomy 16, (8) trisomy 14, and (9) monosomy 1+trisomy 9.

### Statistical Demonstration of the Method

A set of virtual embryos were assembled, a virtual blastomere was biopsied from each embryo, and the ploidy state was determined. The embryo ranking method was then used to rank the embryos, and the rate of expected implantation using the embryo ranking method was compared to the expected implantation when embryos were selected randomly. The ploidy state distributions of the virtual embryos were determined using empirically measured data from both internal and published studies, and the calculated relative probabilities that the embryos have to develop as desired were estimated based on empirical embryo development data.

Data from two published studies, in which 112 embryos were studied both on Day 3 and Day 5 for chromosome copy number using fluorescent in situ hybridization (FISH) technology, (Baart et al., Hum. Reprod., 2006, Vol 21(1), p. 223-233; and Baart et al., Hum Reprod., 2004, Vol 19(3), p. 685-693.) were analyzed to create different groups, and determine the relative development probabilities. Note that the data from these studies was performed with FISH, only 8 chromosomes per cell were analyzed and the ploidy calling on these chromosomes may be expected to have a high error rate. The results were analyzed in order to convert the data into a computable format where each embryo has 205 features. The features were clustered into 2 groups: (1) features at Day 3 such as number of copies of each chromosome, the concordance between results when two cells are analyzed from each embryo, and summary features such as the total number of nullsomies, monosomies, and trisomies observed in each cell; and (2) features at Day 5 such as the percentage of cells that have 0, 1, 2, 3 or 4 copies of each chromosome over the 8 chromosomes measured; the clinical diagnosis at Day 5 of normal or abnormal; and the growth state of the embryos as determined by the number of cells on Day 5 and whether arrested or not.

The Day 3 features were analyzed and the embryos were scored for the likelihood of being euploid on Day 5 after a particular abnormality was observed in one or two biopsied blastomeres on Day 3. The Day 5 features were used as the key outcomes to be modeled and the inputs to the model were the measurements on Day 3. The model was trained using the probability P(D) of embryos in the training dataset being euploid (disomic on the relevant chromosomes across more than 80% of cells analyzed in the blastocyst) on Day 5 after a chromosome was found to be either (1) trisomic in one biopsied cell on Day 3 (P(D/t₁)), (2) trisomic in both biopsied cells on Day 3 (P(D/t₂)), (3) monosomic in one biopsied cell on Day 3 (P(D/m₁)), (4) monosomic in both biopsied cells on Day 3 (P(D/m₂)), (5) nullsomic in one biopsied cell on Day 3 (P(D/n₁)), or (6) nullsomic on both biopsied cells on Day 3 (P(D/n₂)) as described below. Leave-one-out training was used, i.e., the embryo to be scored was left out while the algorithm learned these probabilities. Other methods of training predictive algorithms are well known in the literature, and may equally well be used here. Two alternate approaches were used to learn the probabilities P(D/t₁) . . . P(D/n₂): (1) by ignoring chromosome identity (e.g. chromosome 1, 22, X, etc) and pooling the results over all chromosomes to determine these six probabilities; and (2) in a chromosome specific manner where the probabilities P(D/t₁) . . . P(D/n₂) were learned on a per chromosome basis so that a total of 6×8=48 probabilities were learned. Considered first is the non-chromosome specific model. For the embryo to be scored, the number of chromosomes that were (1) trisomic in one biopsied cell on Day 3 (giving count cₜ₁), (2) trisomic in both biopsied cells (cₜ₂), (3) monosomic in one cell (cₘ₁), (4) monosomic in both cells (cₘ₂), (5) nullisomic in one cell (cₙ₁), and (6) nullisomic in both cells (cₙ₂) were counted. The counts cₜ₁, cₜ₂, cₘ₁, cₘ₂, and Cₙ₂ were used for each embryo and a score, S, was computed for that embryo using the model:

S=(P(D|t₁))^{c}^{ₜ₁}(P(D|t₂))^{c}^{ₜ₂}(P(D|m₁))^{c}^{ₘ₁}(P(D|m₂))^{c}^{ₘ₂}(P(D|n₁))^{c}^{ₙ₁}(P(D|n₂))^{c}^{ₙ₂}

The score S represents the probability that an embryo will be euploid on more than a threshold percentage of cells on Day 5 (for the purposes of the training discussed herein, 80% was used as a threshold) for all chromosomes measured, given the observed counts on Day 3, the learned probabilities from the training dataset, and the simplifying assumption that any chromosomes measured disomic on Day 3 will also be disomic on Day 5. In the case where the probabilities are learned on a chromosome specific manner, the algorithm is similar, except that state of each chromosome is evaluated on Day 3 separately. In this case the state of each chromosomes, of index i, is described the values c_{t1,i}, c_{t2,i}, c_{m1,i}, c_{m2,i}, c_{n1,i}, c_{n2,i} where only one these values is 1, corresponding to the state of the chromosome, and the others are 0. The chromosome specific scores were then combined as follows:

$$S = {\prod }_{i = 1 … 8} {( {P}_{i} ( D \vert {t}_{1} ) )}^{{c}_{t 1 , i}} {( {P}_{i} ( D \vert {t}_{2} ) )}^{{c}_{t 2 , i}} {( {P}_{i} ( D \vert {m}_{1} ) )}^{{c}_{m 1 , i}} {( {P}_{i} ( D \vert {m}_{2} ) )}^{{c}_{m 2 , i}} {( {P}_{i} ( D \vert {n}_{1} ) )}^{{c}_{n 1 , i}} {( {P}_{i} ( D \vert {n}_{2} ) )}^{{c}_{n 2 , i}}$$

To demonstrate whether this embryo ranking method has the potential to improve implantation rates, despite the effects of mosaicism, it was determined whether results of a Day 3 biopsy would improve the probability of selecting normal embryos on Day 5. The design of the simulation was to randomly assign the 112 embryos into 14 virtual families with the number of embryos per family ranging from 5 to 12. For each virtual family, either Day 3 embryos were chosen at random or Day 3 embryos were chosen with the highest score S based on the ranking model. It was then determined whether the chosen embryos were euploid on Day 5, and the rate of normal embryos selected with the rate of normal embryos selected on Day 5 was also determined if the embryos were chosen at random, without ploidy data, from the set of embryos that were morphologically normal on Day 5. For the purposes of this evaluation, the assumption was made that the diagnosis of an embryo as “normal” on Day 5 would be highly correlated with successful implantation.

For each virtual family the estimated improvement in the number of normal embryos selected was then calculated under two scenarios: (1) performing a single cell biopsy on Day 3; (2) performing a two-cell biopsy on Day 3. Since the Baart datasets included biopsies of 2 blastomeres, it was possible to emulate a single cell biopsy by leaving one cell out. Note that in the single cell biopsy scenario, the terms P(D/t₂), P(D/m₂), P(D/n₂) and the corresponding counts cₜ₂, cₘ₂, cₙ₂ are all zero and the model becomes simpler. One thousand simulations were performed, involving assigning the embryos to virtual families and estimating the improvement in rate of normal embryo selection. The mean improvement in rates of selecting normal Day 5 embryos using the model of the present disclosure, as compared to using random selection, is shown in FIG. 4 for both the chromosome-specific model and the non-chromosome specific model. FIG. 5 shows histograms of the improvement in virtual implantation rates for the chromosome specific model and compares the percentage improvement in normal embryo rates on applying the model to a 1-cell biopsy and a 2-cell biopsy. When, using this model system, one cell was biopsied, an improvement of between 50 and 60% in the implantation rates was observed. When two cells per embryo were biopsied, an improvement of between 70% and 80% in the implantation rates was observed.

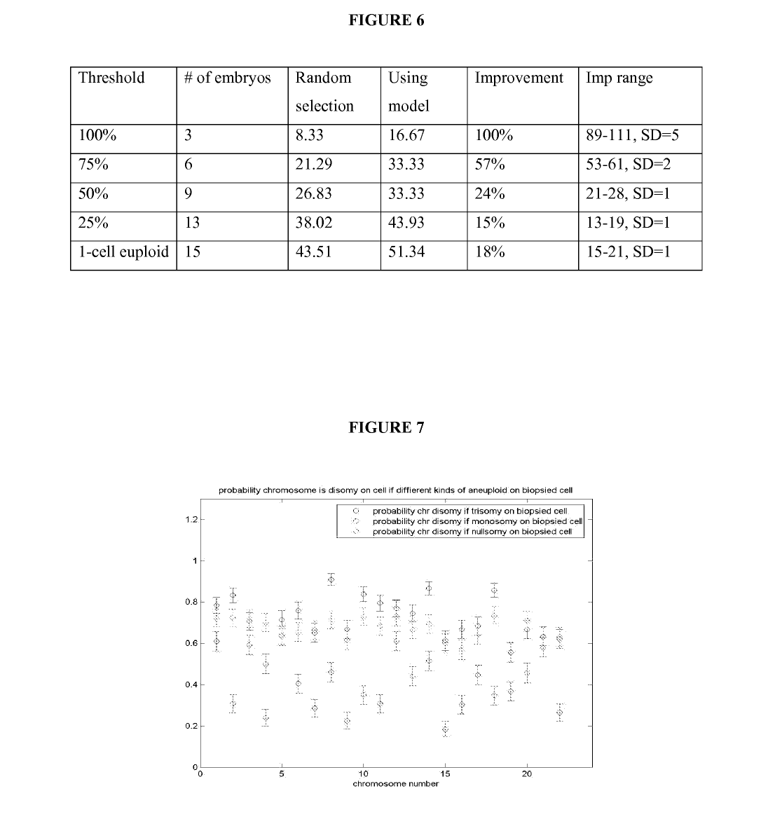

A similar analysis was performed using data collected internally from donated embryos which had been disaggregated and where the ploidy state for each cell had been determined In this case, there were no day 5 outcomes, instead, a surrogate was used, in the form of the euploidy status of the remaining cells after the one blastomere has been biopsied. Since it is not known how many euploid cells are necessary for an embryo to develop as desired, the assumption was made that if a certain fraction of cells among the remaining cells are euploid, then that embryo will develop as desired. Several cutoff thresholds were used for the fraction of cells required for the embryo to be considered one that would develop as desired for the purposes of the surrogate outcome. The results are shown in FIG. 6 where the mean improvement in implantation rates using the model of the present disclosure and internal data, as compared using random selection, is shown. When the threshold was set at 100%, that is, the cell would be considered one which will implant and develop as desired only if 100% of the remaining cells in the virtual embryo are euploid, and only those cells were chosen, then the improvement rate in predicted implantation was 100%. When the threshold was set at 75%, the predicted improvement was 57%; when the threshold was set at 50%, the predicted improvement was 24%; when the threshold was set at 25%, then the predicted improvement was 15%; and when the threshold was that at least one cell in the embryo was euploid, then the predicted improvement was 18%.

In another embodiment, a different simulation was run where the model was trained using model parameters from internal day-3 data and the Baart datasets, and the corresponding 5 outcomes were used for validation. In these simulations, an improvement of 55-60% was consistently measured when selecting a highly ranked embryo as compared to a random selection, where a successful implantation was judged as an embryo that was deemed euploid at day 5.

In another embodiment, to address a shortcoming on the Baart datasets, namely that only eight chromosomes were measured using FISH, and that those measurements are error prone (FISH error rates typically run between 10 and 15%), and the embryos were not grouped into relevant families in the published study, a parallel analysis was performed on internally generated data. These data consisted of measured ploidy data taken from disaggregated blastomeres originating from 27 embryos from 8 different families, where the average number of embryos per family was 3.37, and ranged between 1 and 6. The total number of blastomeres analyzed was 110. The minimum number of blastomeres analyzed per embryo was 2 and the maximum number of blastomeres analyzed per embryo was 8. In this analysis, a single-cell biopsy was assumed and a chromosome-specific model was used as described above. In contrast to the previous analysis, only Day 3 data is analyzed: each of the probabilities Pᵢ(D|t₁), Pᵢ(D|m₁), Pᵢ(D|n₁), represent the likelihood that, given a particular state on the biopsied cell (trisomy, monsomy or nullsomy), another cell chosen from the same embryo will be euploid on that chromosome. One implicit assumption was that embryos that contain at least one euploid cell are more likely to self-correct to euploidy by Day 5 than embryos that do not contain any euploid cells. As in other methods described above, a score was assigned to the embryos, except that this score was computed over all 23 chromosomes:

$$S = {\prod }_{i = 1 … 23} {( {P}_{i} ( D \vert {t}_{1} ) )}^{{c}_{t 1 , i}} {( {P}_{i} ( D \vert {m}_{1} ) )}^{{c}_{m 1 , i}} {( {P}_{i} ( D \vert {n}_{1} ) )}^{{c}_{n 1 , i}}$$

In this case, the score S represents the probability, given the measurement on the biopsied blastomere, that another blastomere taken from the same embryo would be euploid across all chromosomes. This score was use to rank the embryos for each family and the top scoring embryo for each family was chosen for “implantation”. A Day 3 embryo was considered “normal” if that embryo contained one or more fully euploid cells after the single-cell biopsy. One thousand simulations were run and in each simulation a blastomere was chosen at random from each of the embryos in each of the families. If selected at random, the fraction of embryos that contained at least one normal cell was found to be 44.4%. If selected based on the results of the single biopsied cell, the fraction of normal embryos selected was 78.4%, suggesting an improvement in the rate of selection of normal embryos of 76.3%. Leave-one-out training of the model was used.

In order to evaluate the statistical significance of the result over the 27 embryos, the average score S that an embryo received was based on the computed the score for each blastomere that could be biopsied from that embryo; that was computed for each embryo. From that average score, the 27 embryos were ranked. The sum of the ranks of all of the embryos was then computed and compared to expected sum of the ranks if the embryos were randomly ordered. This canonical statistical technique functioned as a way of determining the statistical significance of a ranking method. It was found that the sum of the rank of the embryos using the Day 3 biopsy was improved as compared to the sum of the random ranks with a p-value of 0.0153.

Analysis of the data showed that the improvement in implantation rates is roughly 8% higher when a chromosome-specific model is used. One explanation for this is illustrated in FIG. 7 below where the probabilities Pᵢ(D|t₁), Pᵢ(D|m₁), Pᵢ(D|n₁) for chromosome number i=1 . . . 22 are illustrated. FIG. 7 illustrates the probability of a blastomere in an embryo being diploid on a chromosome if the biopsied cell from that embryo is triploid (blue), monosome (red) or nullisome (green) on that chromosome. The 1-sigma error bar on the estimate of each of these probabilities with limited data is shown. These probabilities vary between chromosomes in a statistically significant manner.

Another example is given here that trains probabilities for 9 bins: trisomy, monosomy, nullisomy: P(D/t), P(D/m), P(D/n); also trisomy of two chromosomes, monosomy of two chromosomes and nullisomy of two chromosomes: P(D/t2), P(D/m2), P(D/n2); and then trisomy+monosomy, trisomy+nullisomy, monosomy+nullisomy: P(D/tm), P(D/tn), P(D/mn). The scoring function (or model) would be:

$${( P ( \frac{D}{t} ) )}^{ct} \times {( P ( \frac{D}{m} ) )}^{cm} \times {( P ( \frac{D}{n} ) )}^{cn} \times {( P ( \frac{D}{t 2} ) )}^{ct 2} \times {( P ( \frac{D}{m 2} ) )}^{cm 2} \times {( P ( \frac{D}{n 2} ) )}^{cn 2} \times {( P ( \frac{D}{tm} ) )}^{ctm} \times {( P ( \frac{D}{tn} ) )}^{ctn} \times {( P ( \frac{D}{mn} ) )}^{cmn}$$

Such a model, with a greater number of bins will allow more accurate probabilities to be computed for: (1) how likely that another cell would be euploid if drawn from same embryo; (2) how likely the embryo is to contain normal cells; (3) how likely the embryo is to be normal on day 5.

### Laboratory Techniques

There are many techniques available allowing the isolation of cells and DNA fragments for genotyping, as well as for the subsequent genotyping of the DNA. The system and method described here can be used in conjunction with any of these techniques, and in many contexts, specifically those involving the isolation of blastomeres from embryos in the context of IVF. This description of techniques is not meant to be exhaustive, and it should be clear to one skilled in the art that there are other laboratory techniques that can achieve the same ends.

### Isolation of Cells

Adult diploid cells can be obtained from bulk tissue or blood samples. Adult diploid single cells can be obtained from whole blood samples using FACS, or fluorescence activated cell sorting. Adult haploid single sperm cells can also be isolated from a sperm sample using FACS. Adult haploid single egg cells can be isolated in the context of egg harvesting during IVF procedures. Isolation of the single cell blastomeres from human embryos can be done using techniques common in in vitro fertilization clinics, such as embryo biopsy.

DNA extraction also might entail non-standard methods for this application. For example, literature reports comparing various methods for DNA extraction have found that in some cases novel protocols, such as the using the addition of N-lauroylsarcosine, were found to be more efficient and produce the fewest false positives.

### Amplification of Genomic DNA

Amplification of the genome can be accomplished by multiple methods including (but not limited to): Polymerase Chain Reaction (PCR), ligation-mediated PCR (LM-PCR), degenerate oligonucleotide primer PCR (DOP-PCR), Whole Genome Amplification (WGA), multiple displacement amplification (MDA), allele-specific amplification, various sequencing methods such as Maxam-Gilbert sequencing, Sanger sequencing, parallel sequencing, sequencing by ligation. The methods described herein can be applied to any of these or other amplification methods.

Background amplification is a problem for each of these methods, since each method would potentially amplify contaminating DNA. Very tiny quantities of contamination can irreversibly poison the assay and give false data. Therefore, it is critical to use clean laboratory conditions, wherein pre- and post-amplification workflows are completely, physically separated. Clean, contamination free workflows for DNA amplification are now routine in industrial molecular biology, and simply require careful attention to detail.

### Genotyping Assay and Hybridization

The genotyping of the amplified DNA can be done by many methods including (but not limited to): molecular inversion probes (MIPs) such as Affymetrix's GENFLEX TAG ARRAY, microarrays such as Affymetrix's 500K array or the ILLUMINA BEAD ARRAYS, or SNP genotyping assays such as AppliedBioscience's TAQMAN assay, other genotyping assays, or fluorescent in-situ hybridization (FISH). The Affymetrix 500K array, MIPs/GENFLEX, TAQMAN and ILLUMINA assay all require microgram quantities of DNA, so genotyping a single cell with either workflow would require some kind of amplification. Each of these techniques has various tradeoffs in terms of cost, quality of data, quantitative vs. qualitative data, customizability, time to complete the assay and the number of measurable SNPs, among others.

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

1. A method for estimating relative likelihoods that each embryo from a set of embryos will develop as desired, wherein at least one cell from each embryo is found to be aneuploid, the method comprising:
determining, on a computer, one or more characteristics of at least one cell from each embryo; and
estimating, on a computer, the relative likelihoods that each embryo will develop as desired, based on the one or more characteristics of the at least one cell for each embryo.

2. (canceled)

3. (canceled)

4. (canceled)

5. The method of claim 1, further comprising selecting at least one embryo from the set of embryos to transfer into a uterus, where the embryo(s) with a relatively higher likelihood of developing as desired is selected.

6. (canceled)

7. (canceled)

8. The method of claim 5, further comprising inserting the selected embryo(s) into a uterus.

9. (canceled)

10. The method of claim 1, wherein the determining step further comprises using an informatics based method to determine the one or more characteristics.

11. (canceled)

12. The method of claim 1, wherein the one or more characteristics comprises a ploidy state.

13. (canceled)

14. (canceled)

15. The method of claim 1, wherein the one or more characteristics is selected from the group consisting of aneuploid, euploid, mosaic, nullsomy, monosomy, uniparental disomy, trisomy, tetrasomy, a type of aneuploidy, unmatched copy error trisomy, matched copy error trisomy, maternal origin of aneuploidy, paternal origin of aneuploidy, a presence or absence of a disease-linked gene, a chromosomal identity of any aneuploid chromosome, an abnormal genetic condition, a deletion or duplication, a likelihood of a characteristic, and combinations thereof, and wherein the one or more characteristics may be associated with a chromosome taken from the group consisting of chromosome one, chromosome two, chromosome three, chromosome four, chromosome five, chromosome six, chromosome seven, chromosome eight, chromosome nine, chromosome ten, chromosome eleven, chromosome twelve, chromosome thirteen, chromosome fourteen, chromosome fifteen, chromosome sixteen, chromosome seventeen, chromosome eighteen, chromosome nineteen, chromosome twenty, chromosome twenty-one, chromosome twenty-two, X chromosome or Y chromosome, and combinations thereof.

16. A method for selecting one or more embryos from a set of embryos for intended insertion into a uterus, the method comprising:
determining, on a computer, at least one characteristic of at least one cell from each embryo in the set of embryos;
determining, on a computer, a relative likelihood that each embryo will develop as desired based on the determined characteristic(s); and
selecting the one or more embryos that are most likely to develop as desired, wherein at least one cell from at least one selected embryo is found to be aneuploid.

17. (canceled)

18. (canceled)

19. (canceled)

20. (canceled)

21. (canceled)

22. (canceled)

23. The method of claim 16, further comprising transferring the one or more selected embryos into a uterus.

24. The method of claim 16, wherein the step of determining the at least one characteristic further comprises using an informatics based method to determine the at least one characteristic.

25. (canceled)

26. (canceled)

27. (canceled)

28. The method of claim 16, wherein the at least one characteristic includes a ploidy state.

29. The method of claim 16 further comprising using the determined characteristic(s) from the at least one cell from the embryo to predict a probability that a plurality of cells, from the embryo, whose at least one characteristic has not been determined are euploid, and where the determination of the relative likelihood that each embryo will develop as desired is based on the predicted probability and the determined characteristic(s).

30. The method of claim 16, wherein the at least one characteristic is selected from the group consisting of aneuploid, euploid, mosaic, nullsomy, monosomy, uniparental disomy, trisomy, tetrasomy, a type of aneuploidy, unmatched copy error trisomy, matched copy error trisomy, maternal origin of aneuploidy, paternal origin of aneuploidy, a presence or absence of a disease-linked gene, a chromosomal identity of any aneuploid chromosome, an abnormal genetic condition, a deletion or duplication, a likelihood of a characteristic, and combinations thereof, and wherein the at least one characteristic may be associated with a chromosome taken from the group consisting of chromosome one, chromosome two, chromosome three, chromosome four, chromosome five, chromosome six, chromosome seven, chromosome eight, chromosome nine, chromosome ten, chromosome eleven, chromosome twelve, chromosome thirteen, chromosome fourteen, chromosome fifteen, chromosome sixteen, chromosome seventeen, chromosome eighteen, chromosome nineteen, chromosome twenty, chromosome twenty-one, chromosome twenty-two, X chromosome or Y chromosome, and combinations thereof.

31. (canceled)

32. (canceled)

33. (canceled)
